(19) United States
(12) Patent Application Publication
(10) Pub. No.: US 2025/0009636 A1
(43) Pub. Date: Jan. 9, 2025

(54) COMPOSITION FOR STABILIZING ACTIVE INGREDIENTS

(71) Applicant: L'OREAL, Paris (FR)

(72) Inventor: Fan WANG, Shanghai (CN)

(73) Assignee: L'OREAL, Paris (FR)

(21) Appl. No.: 18/711,671

(22) PCT Filed: Dec. 10, 2021
(86) PCT No.: PCT/CN2021/137103

§ 371 Date: May 20, 2024

(51) Int. Cl. A61K 8/92; A61Q 19/00

(52) U.S. Cl. CPC A61K 8/92; A61Q 19/00; A61K 2800/51; A61K 2800/522; A61K 2800/591

(57) ABSTRACT

A stabilizing composition comprises: (i) an oil phase containing at least one oil, (ii) at least one fatty alcohol being solid at room temperature, and (iii) at least one antioxidant selected from cinnamic acid derivatives. A combination comprises at least one active ingredient and said stabilizing composition. A non-therapeutic method for caring for and/or making up keratin materials comprises applying said combination to the keratin materials.

### TECHNICAL FIELD

The present invention relates to a stabilizing composition that may improve the stability of at least one active ingredient, and to a non-therapeutic method for caring for and/or making up keratin materials.

### BACKGROUND ART

Skin acts as a natural barrier between internal and external environments and therefore plays an important role in vital biological functions such as protection against mechanical and chemical injury, microorganisms, and ultraviolet damage. The health and appearance of skin, however, can deteriorate due to environmental factors, genetic makeup, nutrition, and sun exposure.

Environmental pollution conditions are fast worsening and becoming more apparent in the daily life of consumers worldwide. The damage of pollution against human skin is also becoming more and more evident. Human skin is also subjected to a variety of insults by extrinsic factors such as ultraviolet (UV) radiation, environmental pollution, wind, heat, infrared radiation, low humidity, harsh surfactants, abrasives, etc. Recent studies suggest that in addition to UV radiation, other environmental factors contribute to the development of solar lentigines, particularly air pollution. Ultimately, these factors result in visible signs of skin damage including small brown patches on the skin, especially in the elderly.

Typical skin damage includes fine lines, wrinkling, hyperpigmentation, sallowness, sagging, dark under-eye circles, puffy eyes, enlarged pores, visible dead skin, i.e., flaking, scaling, dryness, and roughness. Consumers desire to slow the gaining of skin damage and reduce the effects of aging, especially in the face and around the eyes. Radiant and clear skin appears youthful and is a sign of good health and vitality.

Some active ingredients such as ferulic acid, retinol and β-carotene may be beneficial for the youth, health and visual appearance of skin. They are insoluble in water, soluble in an oil and sensitive to water, light, heat and/or oxygen. These active ingredients are instable and exhibit substantial degradation over time. When one or more of them is/are introduced into a cosmetic or dermatological composition for topical application, the degradation of active ingredient is rapid, due to the effect of light, oxygen, metal ions, oxidizing agents, water or, in particular, due to an increase in temperature, preventing the retention of a high level of the active ingredient in the composition during its repeated uses. The degradation of active ingredients negatively influences the efficacy of cosmetic or dermatological composition for improving the skin state. Many efforts have been made to reduce the degradation and improve the stability of these active ingredients. However, some results are not satisfying enough.

Accordingly, there is an ongoing need for a stabilizing composition that may be used to improve the stability of active ingredients and thus increase the efficacy thereof.

### SUMMARY OF THE INVENTION

Therefore, an object of the present invention is to provide a stabilizing composition for improving the stability of at least one active ingredient.

Another object of the present invention is to provide a combination comprising oil-soluble or oil dispersible active ingredients and the stabilizing composition according to the present invention.

Still another object of the present invention is to provide a non-therapeutic method for caring for and/or making up keratin materials.

The inventors have now discovered that the above objects can be achieved by combining the technology of lamellar structured phase and chemically anti-oxidizing technology with selected component(s) to achieve better stability of active ingredients.

Accordingly, in a first aspect, the present invention provides a stabilizing composition, comprising:
(i) an oil phase containing at least one oil,
(ii) at least one fatty alcohol being solid at room temperature, and
(iii) at least one antioxidant selected from cinnamic acid derivatives.

The composition of the present invention may form an oil-in-water emulsion with a lamellar structure (i.e. an alpha-gel structure). Thus, said composition may comprise a continuous aqueous phase and a dispersed oil phase with oil droplets encapsulated within the multi-lamellar interface.

In a second aspect, the present invention provides a combination comprising at least one active ingredient and the stabilizing composition according to the first aspect of the present invention.

In a third aspect, the present invention provides a non-therapeutic method for caring for and/or making up keratin materials, comprising applying the combination according to the second aspect of the present invention to the keratin materials.

Surprisingly, the inventors have found that lamellar structured phase has a synergistic effect with certain antioxidant and optionally chelating agent to improve the stability of active ingredients, such that the combination according to the second aspect of the present invention does not exhibit phase separation and retains at least 85 wt. % of the active ingredients after storage for 8 weeks at 45° C. in a light-resistant glass container, indicating substantially improved stability of active ingredients.

Other advantages of the present invention will emerge more clearly on reading the description and the examples that follow.

### BRIEF DESCRIPTION OF THE DRAWINGS

Implementations of the present invention will now be described, by way of example only, with reference to the attached figure, wherein:

FIG. 1 shows polarized light microscopy image of the sample obtained according to the present invention; and

FIG. 2 shows scanning electron microscopy image of the sample obtained according to the present invention.

### DETAILED DESCRIPTION OF THE INVENTION

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “between . . . and . . . ” and “from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values which are capable of being changed according to the desired purpose as required.

For the purposes of the present invention, the term “keratin materials” is intended to cover human skin, mucous membranes such as the lips. Facial skin is most particularly considered according to the present invention.

All percentages in the present invention refer to weight percentage, unless otherwise specified.

According to an embodiment, the present invention provides a stabilizing composition for improving the stability of at least one active ingredient, comprising:
(i) an oil phase containing at least one oil,
(ii) at least one fatty alcohol being solid at room temperature, and
(iii) at least one antioxidant selected from cinnamic acid derivatives.

### Oil Phase

According to an embodiment of the present invention, the composition of the present invention may comprise an oil phase. Said oil phase comprises at least one oil.

The oil can be volatile or non-volatile.

The term “oil” means a water-immiscible non-aqueous compound that is liquid at room temperature (25° C.) and at atmospheric pressure (760 mmHg). The term “non-volatile oil” means an oil that may remain on keratin materials at room temperature and atmospheric pressure for at least several hours and that especially has a vapour pressure of less than 103 mmHg (0.13 Pa). A non-volatile oil may also be defined as having an evaporation rate such that, under the conditions defined previously, the amount evaporated after 30 minutes is less than 0.07 mg/cm².

According to an embodiment of the present invention, the oil is defined by Hansen Solubility Parameters.

The definition and calculation of the solubility parameters in the Hansen three-dimensional solubility space are described in the article by C. M. Hansen: “The three dimensional solubility parameters”, J. Paint Technol. 39, 105 (1967).

According to this Hansen space:
δ_{d} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.); and
δₐ is determined by the equation: δₐ=(δₚ²+δₕ²)^{1/2}.

The parameters δₚ, δₕ, δ_{d}, and δₐ are expressed in (J/cm³)^{1/2}.

For example, according to one embodiment, if the used active ingredient is retinol, then the oil may have Hansen Solubility Parameters of δ_{d} 16.0-17.0, δₚ 1.5-2.1, and δₕ 4.5-7.0. In a preferable embodiment, the oil may have no unsaturated chemical bonds.

Preferably, the oil is selected from hydrocarbon oils.

The term “hydrocarbon oil” means an oil formed essentially from, or even constituted by, carbon and hydrogen atoms, and optionally O and N atoms, and free of Si and F heteroatoms. Such oil can contain alcohol, ester, ether, carboxylic acid, amine and/or amide groups.

Preferably, the oil is selected from polar hydrocarbon oils such as ester oils, for example, isopropyl myristate, isopropyl palmitate, ethyl hexyl palmitate, triglycerides, isopropyl lauroyl sarcosinate.

As examples of triglycerides, mention can be made of triglycerides of fatty acids containing from 6 to 22 carbon atoms, or preferably from 8 to 18 carbon atoms, such as caprylic/capric triglycerides.

Advantageously, the oil may be present in the composition according to the present invention in an amount ranging from 1 wt. % to 50 wt. %, preferably from 2 wt. % to 30 wt. %, or preferably from 5 wt. % to 20 wt. %, relative to the total weight of the composition.

### Fatty Alcohol

According to an embodiment of the present invention, the composition of the present invention may comprise at least one fatty alcohol being solid at room temperature.

According to an embodiment of the present invention, the fatty alcohol may have a carbon-chain length of C14 and higher, for example a carbon-chain length of C14-C26.

For the purposes of the invention, the fatty alcohol may be linear and saturated.

The fatty alcohol suitable for use in the invention may be selected from myristyl alcohol, pentadecyl alcohol, cetyl alcohol, oleyl alcohol, stearyl alcohol, cetearyl alcohol, and mixtures thereof; or preferably selected from myristyl alcohol, cetyl alcohol, cetearyl alcohol, and mixtures thereof.

As commercial products of myristyl alcohol, mention may be made, for example, of the product sold under the name LANETTE® 14 by the company BASF.

As commercial products of cetyl alcohol, mention may be made, for example, of the products sold under the names Ecorol® 16/98 F and Ecorol® 16/98 P sold by the company Ecogreen Oleochemicals, Tegoalkanol® 16 sold by the company Evonik Goldschmidt, Lanette® 16 sold by the company Cognis, Vegarol® 1698 sold by the company VVF, Alkonat® 1698 P sold by the company Oxiteno, Cetyl Alcohol 98% Min sold by the company Emery Oleochemicals, Ginol® 16 (98%) sold by the company Godrej Industries, Nacol® 16-98 sold by the company Sasol, Kalcol® 6098 sold by the company Kao, and Acilol® 16 sold by the company Aegis Chemical.

As commercial products of cetearyl alcohol, mention may be made, for example, of the products sold under the names Ecorol® 68/50 P and Ecorol® 68/50 P/MB sold by the company Ecogreen Oleochemicals.

Advantageously, the fatty alcohol may be present in the composition according to the present invention in an amount ranging from 2 wt. % to 25 wt. %, preferably from 3 wt. % to 10 wt. %, or preferably from 4 wt. % to 8 wt. %, relative to the total weight of the composition.

### Antioxidant

According to an embodiment of the present invention, the composition of the present invention may comprise at least one antioxidant selected from cinnamic acid derivatives.

According to an embodiment of the present invention, the antioxidant may be a liposoluble antioxidant which is soluble in the oil present in the composition according to the present invention.

The antioxidant may be a derivative of cinnamic acid, e.g., a phenylpropanoid. Phenylpropanoids include: cinnamic acid, caffeic acid, ferulic acid, trans-ferulic acid (including its antioxidant pharmacore 2,6-dihydroxy acetophenome), 5-hydroxyferic acid, sinapic acid, Coumarin, coniferyl alcohol, sinapyl alcohol, eugenol, Chavicol, baicalein, P-coumaric acid and sinapinic acid. Without limiting the mode of action of the present invention, phenylpropanoids can neutralize free radicals.

Amongst others, useful cinnamic acid derivatives may comprise 2-ethylhexyl methoxycinnamate, isopropyl methoxycinnamate, isoamyl methoxycinnamate, diisopropyl methoxycinnamate, pentaerythrityl tetra-di-t-butyl hydroxyhydrocinnamate (Tinogard TT), and/or octadecyl di-t-butyl-4-hydroxyhydrocinnamate.

On the other hand, hindered phenols or semi-hindered phenols may be used as antioxidants according to the present invention. Useful hindered phenols or semi-hindered phenols may comprise: mono- or di- or tri-(a-methylbenzyl)phenol, pentaerythrityl tetra-di-t-butyl hydroxyhydrocinnamate (Tinogard TT), 2,6-di-tert-butyl-4-methylphenol, 2,6-di-tert-butyl-4-ethylphenol, 4,4′-butylidenebis(3-methyl-6-tert-butylphenol) 2,2,-methylenebis(4-ethyl-6-tert-butylphenol), and/or 2,2′-methylenebis(4-methyl-6-tert-butylphenol).

Amongst others, pentaerythrityl tetra-di-t-butyl hydroxyhydrocinnamate is both a derivative of cinnamic acid and a hindered phenol, and is particularly preferred.

Advantageously, the antioxidant is present in the composition according to the present invention in an amount ranging from 0.05 wt. % to 2 wt. %, preferably from 0.08 wt. % to 1 wt. %, or preferably from 0.1 wt. % to 0.5 wt. %, relative to the total weight of the composition.

### Aqueous Phase

According to an embodiment of the present invention, as an oil-in-water emulsion, the stabilizing composition according to the present invention may comprise a continuous aqueous phase.

Said aqueous phase comprises water.

Preferably, the continuous aqueous phase may comprise an organic solvent miscible with water (at room temperature 25° C.) such as polyol having from 2 to 20 carbon atoms, preferably from 2 to 10 carbon atoms, and preferentially having from 2 to 6 carbon atoms, such as ethylene glycol, propylene glycol, butylene glycol, pentylene glycol, hexylene glycol, caprylyl glycol, 1,3-propanediol, diethylene glycol, dipropylene glycol, glycerin, diglycerin, polyethylene glycols; and mixtures thereof.

Advantageously, said aqueous phase may be present in the composition of the present invention in an amount ranging from 40 wt. % to 95 wt. %, preferably from 50 wt. % to 90 wt. %, and or preferably from 55 wt. % to 85 wt. % of the total weight of the composition.

Advantageously, water may be present in the composition of the present invention in an amount ranging from 40 wt. % to 95 wt. %, preferably from 45 wt. % to 90 wt. %, or preferably from 50 wt. % to 80 wt. % relative to the total weight of the composition.

### Chelating Agent

According to an embodiment of the present invention, the composition of the present invention may comprise at least one chelating agent.

Such chelating agents are defined and described in particular in the article “Chelating agents” Kirk Othmer Encyclopedia of Chemical Technology, Vol. 5 pp. 708-739, published in 2003.

The examples of a chelating agent may be made to polyphosphates, aminocarboxylic acids, 1,3-diketones, hydroxycarboxylic acids, polyamines, amino alcohols, heterocyclic aromatic bases, aminophenols, Schiff's bases, tetrapyrroles, sulfur compounds, synthetic macrocyclic compounds, polymers and phosphonic acids.

For the purpose of the present invention, useful chelating agents may comprise ethylenediamine tetraacetic acid (EDTA), aminotriacetic acid, diethylene triaminepentaacetic acid, and a salt thereof, e.g., N, N-bis(carboxymethyl) glutamic acid, disodium EDTA, tetrasodium EDTA, tetrasodium salt of N,N-bis(carboxymethyl) glutamic acid (glutamic acid diacetic acid, GLDA); hydroxyl carboxylic acids, e.g., citric acid, tartaric acid, glucuronic acid, succinic acid, ethylenediamine disuccinic acid (EDDS), and a salt thereof; hydroxyl aminocarboxylic acids, e.g., hydroxyethylethylenediamine triacetic acid (HEDTA), dihydroxyethylglycine (DEG), and a salt thereof; polyphosphonic acid, and a salt thereof; other phosphor-containing organic acid, e.g., phytic acid, and a salt thereof, e.g., sodium phytate, potassium phytate polycarboxylic acid, e.g., polyacrylic acid, polymethacrylic acid, and a salt thereof.

For example, a useful chelating agent may be chosen from sodium citrate, disodium EDTA, tetrasodium EDTA, tetrasodium GLDA, trisodium EDDS, sodium phytate, potassium phytate, and mixtures thereof.

According to the present invention, the chelating agent may be present in the composition of the present in an amount from 0.001 wt. % to 1 wt. %, preferably from 0.01 wt. % to 0.5 wt. %, or preferably from 0.05 wt. % to 0.2 wt. %, relative to the total weight of the composition according to the present invention.

### Surfactant

According to an embodiment of the present invention, the composition of the present invention may comprise at least one surfactant.

The surfactant is not particularly limited and it can be selected from anionic surfactants, amphoteric surfactants, nonionic surfactants, and cationic surfactants. Preferably, the surfactant is selected from anionic surfactants and nonionic surfactants.

### Anionic Surfactant

The term “anionic surfactant” means a surfactant comprising, as ionic or ionizable groups, only anionic groups.

In the present description, a species is termed as being “anionic” when it bears at least one permanent negative charge or when it can be ionized as a negatively charged species, under the conditions of use of the composition of the invention (for example the medium or the pH) and not comprising any cationic charge.

The anionic surfactants may be sulfate, sulfonate and/or carboxylic (or carboxylate) surfactants. Needless to say, a mixture of these surfactants may be used.

It is understood in the present description that:
carboxylate anionic surfactants comprise at least one carboxylic or carboxylate function (—COOH or —COO⁻) and may optionally also comprise one or more sulfate and/or sulfonate functions;
the sulfonate anionic surfactants comprise at least one sulfonate function (—SO₃H or —SO₃) and may optionally also comprise one or more sulfate functions, but do not comprise any carboxylate functions; and
the sulfate anionic surfactants comprise at least one sulfate function but do not comprise any carboxylate or sulfonate functions.

The carboxylic anionic surfactants that may be used thus comprise at least one carboxylic or carboxylate function (—COOH or —COO⁻).

They may be chosen from the following compounds: acylglycinates, acyllactylates, acylsarcosinates, acylglutamates; alkyl-D-galactosideuronic acids, carboxylates such as stearates; alkyl ether carboxylic acids, alkyl(C6-30 aryl) ether carboxylic acids, alkylamido ether carboxylic acids; and also the salts of these compounds;
the alkyl and/or acyl groups of these compounds comprising from 6 to 30 carbon atoms, especially from 12 to 28, better still from 14 to 24 or even from 14 to 18 carbon atoms; the aryl group preferably denoting a phenyl or benzyl group;
these compounds possibly being polyoxyalkylenated, especially polyoxyethylenated, and then preferably comprising from 1 to 50 ethylene oxide units and better still from 2 to 10 ethylene oxide units.

Use may also be made of the C6-C24 alkyl monoesters of polyglycoside-polycarboxylic acids, such as C6-C24 alkyl polyglycoside-citrates, C6-C24 alkyl polyglycoside-tartrates and C6-C24 alkyl polyglycoside-sulfosuccinates, and salts thereof.

Among the above carboxylic surfactants, mention may be made particularly of polyoxyalkylenated alkyl(amido) ether carboxylic acids and salts thereof, in particular those comprising from 2 to 50 alkylene oxide and in particular ethylene oxide groups, such as the compounds sold by the company Kao under the name Akypo,

The polyoxyalkylenated alkyl (amido) ether carboxylic acids that may be used are preferably chosen from those of formula (1):

R₁ [symbol] OC₂H₄)ₙ—OCH₂COOA (1)

in which:
R1 represents a linear or branched C6-C24 alkyl or alkenyl radical, an alkyl(C8-C9)phenyl radical, a radical R2CONH—CH2-CH2- with R2 denoting a linear or branched C9-C21 alkyl or alkenyl radical,

preferably, R1 is a C8-C20 and preferably C8-C18 alkyl radical, and aryl preferably denotes phenyl,
n is an integer or decimal number (average value) ranging from 2 to 24 and preferably from 2 to 10,
A denotes H, ammonium, Na, K, Li, Mg or a monoethanolamine or triethanolamine residue.

It is also possible to use mixtures of compounds of formula (1), in particular mixtures of compounds containing different groups R1.

The polyoxyalkylenated alkyl(amido) ether carboxylic acids that are particularly preferred are those of formula (1) in which:
R1 denotes a C12-C14 alkyl, cocoyl, oleyl, nonylphenyl or octylphenyl radical,
A denotes a hydrogen or sodium atom, and
n varies from 2 to 20 and preferably from 2 to 10.

Preferentially, use is made of compounds of formula (1) in which R denotes a C12 alkyl radical, A denotes a hydrogen or sodium atom and n ranges from 2 to 10.

Preferentially, the carboxylic anionic surfactants are chosen, alone or as a mixture, from:
acylglutamates, especially of C6-C24 or even C12-C20, such as stearoylglutamates, and in particular disodium stearoylglutamate, sodium stearoylglutamate;
acylsarcosinates, especially of C6-C24 or even C12-C20, such as palmitoylsarcosinates, and in particular sodium palmitoylsarcosinate;
acyllactylates, especially of C12-C28 or even C14-C24, such as behenoyllactylates, and in particular sodium behenoyllactylate;
C6-C24 and especially C12-C20 acylglycinates;
(C6-C24)alkyl ether carboxylates and especially (C12-C20)alkyl ether carboxylates;
polyoxyalkylenated (C₆-C₂₄)alkyl(amido) ether carboxylic acids, in particular those comprising from 2 to 50 ethylene oxide groups;
in particular in the form of alkali metal or alkaline-earth metal, ammonium or amino alcohol salts.

The sulfonate anionic surfactants that may be used comprise at least one sulfonate function (—SO₃H or —SO₃⁻).

They may be chosen from the following compounds: alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds;
the alkyl groups of these compounds comprising from 6 to 30 carbon atoms, especially from 12 to 28, better still from 14 to 24 or even from 14 to 18 carbon atoms; the aryl group preferably denoting a phenyl or benzyl group;
these compounds possibly being polyoxyalkylenated, especially polyoxyethylenated, and then preferably comprising from 1 to 50 ethylene oxide units and better still from 2 to 10 ethylene oxide units.

Preferentially, the sulfonate anionic surfactants are chosen, alone or as a mixture, from:
C6-C24 and especially C12-C20 acyltaurates, especially sodium methyl stearoyl taurate;
C6-C24 and especially C12-C20 alkylsulfosuccinates, especially laurylsulfosuccinates;
C6-C24 and especially C12-C20 alkyl ether sulfosuccinates;
(C6-C24) acylisethionates and preferably (C12-C18) acylisethionates,
in particular in the form of alkali metal or alkaline-earth metal, ammonium or amino alcohol salts.

The sulfate anionic surfactants that may be used comprise at least one sulfate function (—OSO₃H or —OSO₃⁻).

They may be chosen from the following compounds: alkyl sulfates, alkyl ether sulfates, alkylamido ether sulfates, alkylaryl polyether sulfates, monoglyceride sulfates; and also the salts of these compounds;
the alkyl groups of these compounds comprising from 6 to 30 carbon atoms, especially from 12 to 28, better still from 14 to 24 or even from 14 to 18 carbon atoms; the aryl group preferably denoting a phenyl or benzyl group;
these compounds possibly being polyoxyalkylenated, especially polyoxyethylenated, and then preferably comprising from 1 to 50 ethylene oxide units and better still from 2 to 10 ethylene oxide units.

Preferentially, the sulfate anionic surfactants are chosen, alone or as a mixture, from:
alkyl sulfates, especially of C6-C24 or even C12-C20,
alkyl ether sulfates, especially of C6-C24 or even C12-C20, preferably comprising from 2 to 20 ethylene oxide units;
in particular in the form of alkali metal or alkaline-earth metal, ammonium or amino alcohol salts.

When the anionic surfactant is in salt form, the said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferentially, the anionic surfactants are chosen, alone or as a mixture, from:
C6-C24 and especially C12-C20 alkyl sulfates;
C6-C24 and especially C12-C20 alkyl ether sulfates; preferably comprising from 2 to 20 ethylene oxide units;
C6-C24 and especially C12-C20 alkylsulfosuccinates, especially laurylsulfosuccinates;
C6-C24 and especially C12-C20 alkyl ether sulfosuccinates;
(C6-C24) acylisethionates and preferably (C12-C18) acylisethionates;
C6-C24 and especially C12-C20 acylsarcosinates; especially palmitoylsarcosinates;
(C6-C24)alkyl ether carboxylates, preferably (C12-C20)alkyl ether carboxylates;
polyoxyalkylenated (C6-C24)alkyl(amido) ether carboxylic acids and salts thereof, in particular those comprising from 2 to 50 alkylene oxide and in particular ethylene oxide groups;
C6-C24 and especially C12-C20 acyltaurates;
C6-C24 and especially C12-C20 acylglutamates;
C12-C20 carboxylates;
C6-C24 and especially C12-C20 acylglycinates;
in particular in the form of alkali metal or alkaline-earth metal, ammonium or amino alcohol salts.

### Nonionic Surfactant

They may be chosen from alcohols, α-diols and (C₁₋₂₀) alkylphenols, these compounds being polyethoxylated and/or polypropoxylated and/or polyglycerolated, the number of ethylene oxide and/or propylene oxide groups possibly ranging from 1 to 100, and the number of glycerol groups possibly ranging from 2 to 30; or alternatively these compounds comprising at least one fatty chain comprising from 8 to 30 carbon atoms and especially from 16 to 30 carbon atoms.

Mention may also be made of condensates of ethylene oxide and of propylene oxide with fatty alcohols; polyethoxylated fatty amides preferably having from 2 to 30 ethylene oxide units, polyglycerolated fatty amides comprising on average from 1 to 5, and in particular from 1.5 to 4, glycerol groups; polyethoxylated fatty acid esters of sorbitan preferably containing from 2 to 40 ethylene oxide units, fatty acid esters of sucrose, polyoxyalkylenated and preferably polyoxyethylenated fatty acid esters containing from 2 to 150 mol of ethylene oxide, including oxyethylenated plant oils, N—(C6-24 alkyl) glucamine derivatives, amine oxides such as (C10-14 alkyl)amine oxides or N—(C10-14 acyl)aminopropylmorpholine oxides.

Mention may also be made of nonionic surfactants of alkyl(poly)glycoside type, represented especially by the following general formula:

R₁O—(R₂O)ₜ-(G)ᵥ

in which:
R1 represents a linear or branched alkyl or alkenyl radical comprising 6 to 24 carbon atoms and especially 8 to 18 carbon atoms, or an alkylphenyl radical whose linear or branched alkyl radical comprises 6 to 24 carbon atoms and especially 8 to 18 carbon atoms;
R2 represents an alkylene radical comprising 2 to 4 carbon atoms,
G represents a sugar unit comprising 5 to 6 carbon atoms,
t denotes a value ranging from 0 to 10 and preferably 0 to 4,
v denotes a value ranging from 1 to 15 and preferably 1 to 4.

Preferably, the alkylpolyglycoside surfactants are compounds of the formula described above in which:
R1 denotes a linear or branched, saturated or unsaturated alkyl radical comprising from 8 to 18 carbon atoms,
R2 represents an alkylene radical comprising 2 to 4 carbon atoms,
t denotes a value ranging from 0 to 3 and preferably equal to 0,
G denotes glucose, fructose or galactose, preferably glucose;
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. C8/C16 alkyl(poly)glycosides 1,4, and especially decyl glucosides, caprylyl/capryl glucosides, and coco-glucoside are most particularly preferred.

Among the commercial products, mention may be made of the products sold by the company COGNIS under the names PLANTAREN® (600 CS/U, 1200 and 2000) or PLANTACARE® (818, 1200 and 2000); the products sold by the company SEPPIC under the names ORAMIX CG 110 and ORAMIX NS 10; the products sold by the company BASF under the name LUTENSOL GD 70, or else the products sold by the company CHEM Y under the name AG10 LK.

Preferably, use is made of C8/C16-alkyl(poly)glycosides 1,4, especially as an aqueous 53% solution, such as those sold by Cognis under the reference Plantacare® 818 UP.

Preferentially, the nonionic surfactants are chosen from C6-C24 alkyl polyglycosides, and more particularly C8-C18 alkyl(poly)glycosides, polyethoxylated C8-C30 fatty acid esters of sorbitan, polyethoxylated C8-C30 fatty alcohols and polyoxyethylenated C8-C30 fatty acid esters, these compounds preferably containing from 2 to 150 mol of ethylene oxide, and mixtures thereof.

Preferably, the surfactant is selected from:
C12-C20 acylglutamates, such as disodium stearoylglutamate, sodium stearoylglutamate;
C12-C20 acyltaurates, especially sodium methyl stearoyl taurate;
C14-18 carboxylates such as stearates, especially sodium stearate;
C8-C18 alkyl(poly)glycosides, such as coco-glucoside;
polyethoxylated fatty acid esters of sorbitan containing from 2 to 40 ethylene oxide units, such as polysorbate 60;
polyethoxylated C8-C30 fatty alcohols containing from 2 to 40 ethylene oxide units, such as steareth-2; and
a mixture thereof.

Advantageously, the surfactant may be present in the composition according to the present invention in an amount ranging from 0.05 wt. % to 5 wt. %, preferably from 0.1 wt. % to 3 wt. %, relative to the total weight of the composition.

### Active Ingredient

According to an embodiment of the present invention, the combination of the present invention may comprise an active ingredient which is soluble in the oil present in the composition according to the present invention.

Optionally, the oil-soluble or oil-dispersible active ingredient is sensitive to water, light, heat and/or oxygen.

As used herein, “active ingredient is sensitive to water, light, heat and/or oxygen” means that the active ingredient will degrade in contact with water, light, heat and/or oxygen.

For example, the oil-soluble or oil-dispersible active ingredient may be selected from retinol (vitamin A) and derivatives thereof, such as retinyl palmitate, retinyl linoleate, retinal, and hydroxypinacolone retinoate.

For the purposes of the present invention, the term “retinol” denotes all the isomers of retinol, notably all-trans retinol, 13-cis retinol, 11-cis retinol and 9-cis retinol, but also 3,4-didehydroretinol.

According to a particular embodiment, retinol and/or derivatives thereof may be introduced into the composition in a form which is dissolved in an oil, such as a plant oil, for example soybean oil, notably in a content ranging from 5.0% to 20% by weight, preferably of about 10% by weight in the oil.

Advantageously, the oil-soluble or oil-dispersible active ingredient may be present in the composition according to the present invention in an amount ranging from 0.01 wt. % to 3 wt. %, preferably from 0.05 wt. % to 1 wt. %, or preferably from 0.1 wt. % to 0.2 wt. %, relative to the total weight of the composition.

### Additional Active Component

The combination of the present invention may comprise an active component in addition to the oil-soluble or oil-dispersible active ingredient mentioned above.

As examples of the active component, mention can be made of natural extracts; vitamins such as vitamin B5 (panthenol), vitamin B3 (niacinamide), and derivatives of said vitamins (in particular esters) and mixtures thereof; urea; caffeine; salicylic acid and derivatives thereof; alpha-hydroxyacids such as lactic acid or glycolic acid and derivatives thereof; sunscreens; extracts from algae, fungi, plants, yeasts and bacteria; enzymes; agents acting on the microcirculation, and mixtures thereof.

The skilled in the art can select the amount of the additional active component based on the final use of the composition according to the present invention.

### Adjuvant or Additive

The combination of the present invention may also comprise conventional adjuvants or additives, for instance fragrances, preserving agents (such as, chlorphenesin and phenoxyethanol) and bactericides, solubilizing agents (such as, ethanol, butanol, butylene glycol, propylene glycol, and dipropylene glycol), thickeners (such as xanthan gum, acrylamide/sodium acryloyldimethyltaurate copolymer, acrylates/C10-30 alkyl acrylate crosspolymer, hydroxypropyl guar; oil thickeners such as polyurethane, polyamide, ethylcellulose), pH regulators (such as, triethanolamine, citric acid and sodium hydroxide), dyes/pigments (such as, metal oxide pigments, organic pigments, and organic lakes), fillers (such as, aluminum starch octenylsuccinate and polymethiylsisesquioxane) and mixtures thereof.

The skilled in the art can select the amount of the additional adjuvants or additive so as not to adversely impact the final use of the composition according to the present invention.

According to a particularly preferred embodiment, the present invention provides a stabilizing composition in the form of an oil-in-water emulsion with a lamellar structure for improving the stability of active ingredients comprising, relative to the total weight of the composition:
(i) from 1 wt. % to 50 wt. % of an oil phase containing at least one oil having Hansen Solubility Parameters of d 16.0-17.0, p 1.5-2.1, and h 4.5-7.0 and having no unsaturated chemical bonds,
(ii) from 2 wt. % to 25 wt. % of at least one fatty alcohol being solid at room temperature and having a carbon-chain length of 14 and higher,
(iii) from 0.05 wt. % to 2 wt. % of at least one antioxidant selected from cinnamic acid derivatives,
(iv) from 0.001 wt. % to 1 wt. % of at least one chelating agent, and
(v) from 0.05 wt. % to 5 wt. % of at least one surfactant.

According to another particularly preferred embodiment, the present invention provides a combination comprising, relative to the total weight of the combination:
(a) from 0.01 wt. % to 3 wt. % of retinol and/or derivatives thereof;
(b) a stabilizing composition comprising, relative to the total weight of the combination:
(i) from 1 wt. % to 50 wt. % of an oil phase containing at least one oil having Hansen Solubility Parameters of d 16.0-17.0, p 1.5-2.1, and h 4.5-7.0 and having no unsaturated chemical bonds,
(ii) from 2 wt. % to 25 wt. % of at least one fatty alcohol being solid at room temperature and having a carbon-chain length of 14 and higher,
(iii) from 0.05 wt. % to 2 wt. % of at least one antioxidant selected from cinnamic acid derivatives,
(iv) from 0.001 wt. % to 1 wt. % of at least one chelating agent, and
(v) from 0.05 wt. % to 5 wt. % of at least one surfactant.

### Galenic Form, Use and Method

The stability composition and the combination comprising at least one active ingredient and the stability composition according to the present invention may be in the form of an oil-in-water emulsion.

The stability composition and the combination comprising at least one active ingredient and the stability composition according to the present invention may have a lamellar structure or a lamellar structured phase.

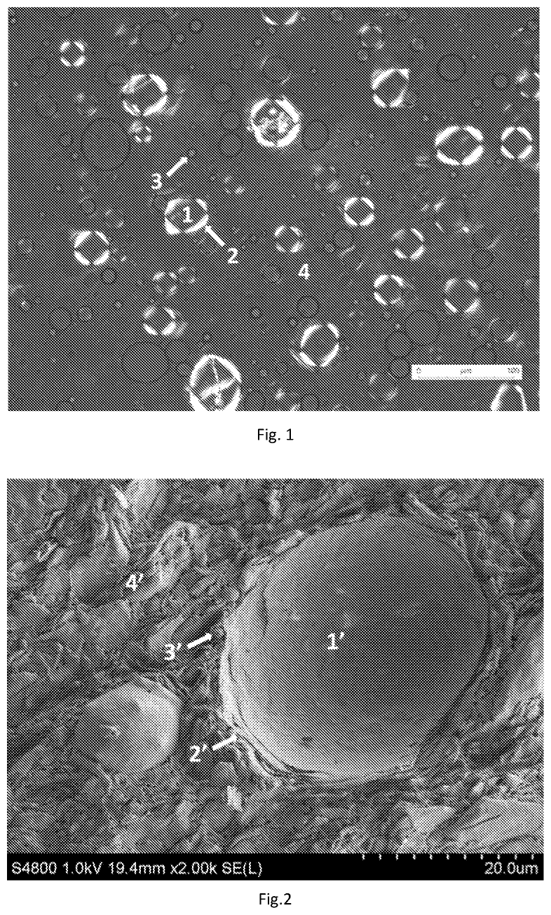

The term “lamellar structure” or “lamellar structured phase” means a liquid crystal structure, or a swollen or non-swollen crystalline lamellar hydrate phase with plane symmetry, comprising several amphiphilic bilayers arranged in parallel and separated by a liquid medium which is generally water. Lamellar structure has a characteristic optical effect when observed with a light microscope under 90° cross polarized light with unique optical effects, as shown in FIG. 1. If the unique optical effect can be observed, it means that the lamellar structure is formed.

The combination according to the second aspect of the present invention can be used for caring for and/or making up keratin materials.

According to an embodiment, the present invention provides a non-therapeutic method for caring for and/or making up keratin materials, comprising applying the combination according to the second aspect of the present invention to the keratin materials.

The following examples serve to illustrate the present invention without, however, being limiting in nature.

### EXAMPLES

Main raw materials used, trade names and supplier thereof are listed in Table 1.

TABLE 1
Raw material information

| INCI Name | Trade Name | Supplier |
|---|---|---|
| Glycerin | CremerGLYC Refined | CREMER OLEO (IOI) |
| | Glycerine 99.7% RSPO MB | |
| Caprylic/Capric Triglyceride | WILFARESTER ™ MCT | WILMAR |
| | 7030 RSPO MB | |
| Cetearyl Alcohol | ECOROL ® 68/50 P | ECOGREEN |
| | | OLEOCHEMICALS |
| Retinol | RETINOL 10SU | BASF |
| Sodium Methyl Stearoyl | Nikkol SMT | NIKKO |
| Taurate | | |
| Isopropyl Myristate | PALMESTER ® 1512/MB | KLK OLEO |
| Xanthan Gum | KELTROL ® CG-T | CP KELCO |
| Pentaerythrityl | TINOGARD ® TT | BASF |
| Tetra-Di-T-Butyl | | |
| Hydroxyhydrocinnamate | | |
| Trisodium Ethylenediamine | NATRLQUEST ® E30 | INNOSPEC ACTIVE |
| Disuccinate | | CHEMICALS |
| Tetrasodium Glutamate | DISSOL VINE ® GL-47-S | AKZO NOBEL |
| Diacetate | | (NOURYON) |
| Citric Acid | CITRIC ACID | RZBC |
| | MONOHYDRATE | |
| | BP/USP/FCC/E330 | |
| Cocos Nucifera (Coconut) Oil | HUILE DE COPRAH | SIO (ADM) |
| | RAFFINEE GV 24/26 | |
| Octyldodecanol | EUTANOL ® G-JP | BASF |
| Glycine Soja (Soybean) Oil | REFINED CT SOYBEAN | ZOR |
| | OIL | |
| Isopropyl Lauroyl Sarcosinate | ELDEW ® SL-205 | AJINOMOTO |

### Examples 1-2 and Comparative Examples 1-7

Samples of examples (Ex.) 1-2 and comparative examples (CE.) 1-7 were prepared according to the amounts given in Tables 2 and 3, respectively. The amount of each component was given in wt. % of the active material relative to the total weight of the composition containing it.

TABLE 2
Samples of examples 1-2

| Components | Ex. 1 | Ex. 2 |
|---|---|---|
| Glycerin | 20.00 | 20.00 |
| Caprylic/Capric Triglyceride | 10.00 | 10.00 |
| Cetearyl Alcohol | 4.00 | 4.00 |
| Retinol | 0.20 | 0.20 |
| Glycine Soja (Soybean) Oil / Glycine Soja Oil | 1.80 | 1.80 |
| Sodium Methyl Stearoyl Taurate | 0.56 | 0.56 |
| Xanthan Gum | 0.10 | |
| Pentaerythrityl Tetra-Di-T-Butyl | 0.10 | 0.10 |
| Hydroxyhydrocinnamate | | |
| Tetrasodium Glutamate Diacetate | 0.15 | 0.15 |
| Water | qs 100 | qs 100 |
| pH | 6.5 | 6.5 |

TABLE 3-1
Samples of comparative examples 1-3

| Components | CE. 1 | CE. 2 | CE. 3 |
|---|---|---|---|
| Glycerin | 20.00 | 20.00 | 20.00 |
| Caprylic/Capric Triglyceride | 10.00 | 10.00 | 10.00 |
| Cetearyl Alcohol | 4.00 | | |
| Retinol | 0.20 | 0.20 | 0.20 |
| Glycine Soja (Soybean) Oil/ | 1.80 | 1.80 | 1.80 |
| Glycine Soja Oil | | | |
| Sodium Methyl Stearoyl Taurate | 0.56 | 0.78 | 0.78 |
| Xanthan Gum | | 2.00 | 2.00 |
| Pentaerythrityl Tetra-Di-T-Butyl | | | 0.10 |
| Hydroxyhydrocinnamate | | | |
| Tetrasodium Glutamate Diacetate | | | 0.15 |
| Water | qs 100 | qs 100 | qs 100 |
| pH | 6.5 | 6.5 | 6.5 |

TABLE 3-2
Samples of comparative examples 4-7

| Components | CE. 4 | CE. 5 | CE. 6 | CE. 7 |
|---|---|---|---|---|
| Glycerin | 20.00 | 20.00 | 20.00 | 20.00 |
| Glycine Soja (Soybean) Oil | 10.00 | | | |
| Cetearyl Alcohol | 4.00 | 4.00 | 4.00 | 4.00 |
| Retinol | 0.20 | 0.20 | 0.20 | 0.20 |
| Glycine Soja (Soybean) Oil/ | 1.80 | 1.80 | 1.80 | 1.80 |
| Glycine Soja | | | | |
| Oil | | | | |
| Sodium Methyl Stearoyl Taurate | 0.56 | 0.56 | 0.56 | 0.56 |
| Cocos Nucifera (Coconut) Oil | | | | 10.00 |
| Octyldodecanol | | | 10.00 | |
| Caprylic/Capric Triglyceride | | | | |
| Xanthan Gum | | | | |
| Isopropyl Lauroyl Sarcosinate | | 10.00 | | |
| Water | qs 100 | qs 100 | qs 100 | qs 100 |
| pH | 6.5 | 6.5 | 6.5 | 6.5 |

Samples of examples 1-2 represented compositions or combinations according to the present invention, which formed an oil-in-water emulsion with a lamellar structured phase and comprised an antioxidant.

Sample of comparative example 1 formed an oil-in-water emulsion with a lamellar structured phase, but did not comprise any antioxidant.

Sample of comparative example 2 formed an oil-in-water emulsion without a lamellar structured-phase, and did not comprise any antioxidant.

Sample of comparative example 3 formed an oil-in-water emulsion without a lamellar structured-phase, and comprised an antioxidant.

Samples of comparative examples 4-7 did not comprise any antioxidant, and comprised undesirable oil types.

### Preparation Process

Samples of examples listed above were prepared as follows, taking the preparation of example 1 as an example:
1). heating the oil phase that contains cetearyl alcohol, caprylic/capric triglyceride, pentaerythrityl tetra-di-t-butyl hydroxyhydrocinnamate, and other oil soluble components at 75° C. till oil soluble components were fully dissolved; and heating the water phase that contains water, glycerin, Sodium Methyl Stearoyl Taurate, chelating agent, and other water soluble components in another vessel until water soluble components were fully dissolved;
2). adding the oil phase to the water phase and homogenizing upon heating at 75° C. till an even emulsion was formed; and
3). cooling with gentle mixing till RT to form a cream;
4). removing air bubbles from the cream; and
5). mixing retinol pre-dissolved or -dispersed in soybean oil with the protection of nitrogen into the cream until evenly dispersed.

### Evaluation

Whether a lamellar structure is formed in each sample obtained was evaluated as follows.

Samples was observed using a Leica DLMB microscope under 90° cross polarized light, and microscopic pictures were taken. If characteristic optical effects of lamellar structure were observed, it means that lamellar structure was formed.

FIG. 1 shows polarized light microscopy image of the sample obtained according to the present invention, wherein, 1 represents an oil droplet encapsulated within the multi-lamellar interface, 2 represents the multi-lamellar interface, 3 represents dispersed oil with retinol and 4 represents the lamellar continuous water phase. Spherical structures are dispersed oil phases, which are surrounded by the multi-lamellar interfaces.

Similar photos were obtained for samples of examples 1-2 and comparative example 1.

FIG. 2 shows scanning electron microscopy image of the sample obtained according to the present invention, wherein, 1′ represents dispersed oil phase, 2′ represents the multi-lamellar interface, 3′ represents dispersed oil droplet with retinol and 4′ represents the lamellar continuous water phase.

Similar photos were obtained for samples of examples 1-2 and comparative example 1.

It can be seen that a lamellar structure is formed in samples of examples 1-2 and comparative example 1.

### Retinol Degradation at 45° C. for 2 Month

The retinol content in each sample obtained was analyzed using a Thermo Fisher UltiMate 3000 high performance liquid chromatography (UPLC) unit. Before subjected to injection, samples were mixed evenly in their original containers and 900-1000 mg were sampled with a glass dropped and dissolved in 25 mL of isopropyl alcohol. Samples were stilled for 30 minutes then filtered for further HPLC analysis. The HPLC unit was equipped with an ACQUITY UPLC BEH Shield RP18, 100×2.1 mm, 1.7 μm column set at 60° C. The sample injection volume was 1 μL. The mobile phase used was MeOH/H₂O with 0.2% H₃PO₄ with gradient elusion described in Table 4. A diode array detector was used and retinol was detected at the wavelength of 325 nm, and the signature elution peak of retinol was at 2.14 min. A calibration curve with retinol content from 5 ppm to 100 ppm was used to calculate the final retinol content in a sample.

TABLE 4
Gradient elution profile

| Time, min | Flow rate, mL/min | MeOH, % | H₂O (+H₃PO₄ 0.2%), % |
|---|---|---|---|
| 0 | 0.6 | 70 | 30 |
| 3.50 | 0.6 | 100 | 0 |
| 4.50 | 0.6 | 100 | 0 |
| 5.50 | 0.6 | 85 | 15 |
| 5.51 | 0.6 | 70 | 30 |
| 6.50 | 0.6 | 70 | 30 |

The retinol degradation at 45° C. for 2 months was calculated according to the following equation:

(C_{T0}−C_{T2M})/C_{T0}*100%

wherein
C_{T0} indicates the content of retinol of the sample freshly made;
C_{T2M} indicates the content of retinol of the sample after being still at 45° C. for 2 month.

The accuracy of the quantification methods is ±5%.

The results of retinol degradation at 45° C. for 2 months of each composition prepared above were listed in Tables 5-6.

TABLE 5
Retinol degradation after 2 months of storage at 45° C., examples

| Properties | Ex. 1 | Ex. 2 |
|---|---|---|
| Retinol degradation | 10% | 10% |

TABLE 6
Retinol degradation after 2 months of
storage at 45° C., comparative examples

| Properties | CE. 1 | CE. 2 | CE. 3 | CE. 4 | CE. 5 | CE. 6 | CE. 7 |
|---|---|---|---|---|---|---|---|
| Retinol degradation | 25% | 30% | 20% | 100% | 65% | 75% | 85% |

It can be seen that samples of examples 1-2, which formed a lamellar structured phase and comprised an antioxidant, exhibit substantially improved stability relative to comparative examples 1-7. The comparison of Ex. 1-2 vs. CE. 1 and CE.3 vs. CE.2 clearly reveals the positive effects in the stabilizing of retinol achieved by the addition of antioxidant. By comparing Ex. 1-2 vs. CE. 3 and CE.1 vs. CE.2, it can be seen that, relative to an non-structured emulsion, the formation of an lamellar structured emulsion provides retinol with an even better stability, no matter in the presence or absence of antioxidant. The results of CE.4-7 show the deterioration in the stability of retinol due to the use of undesirable oil types. These results demonstrate the synergistic effect in the improvement of the stability of retinol by combining lamellar structured phase technology with chemically anti-oxidizing technology.

1. A stabilizing composition, comprising:
(i) an oil phase containing at least one oil,
(ii) at least one fatty alcohol being solid at room temperature, and
(iii) at least one antioxidant selected from cinnamic acid derivatives.

2. The stabilizing composition according to claim 1, wherein the oil has Hansen Solubility Parameters of δd 16.0-17.0, δp 1.5-2.1, and δh 4.5-7.0 and has no unsaturated chemical bonds.

3. The stabilizing composition according to claim 1, wherein the oil is selected from the group consisting of isopropyl myristate, isopropyl palmitate, ethyl hexyl palmitate, triglycerides, isopropyl lauroyl sarcosinate, and mixtures thereof.

4. The stabilizing composition according to claim 1, wherein the oil is present in an amount ranging from 1 wt. % to 50 wt. %, relative to the total weight of the stabilizing composition.

5. The stabilizing composition according to claim 1, wherein the fatty alcohol has a carbon-chain length of C14-C26.

6. The stabilizing composition according to claim 1, wherein the fatty alcohol is present in an amount ranging from 2 wt. % to 25 wt. %, relative to the total weight of the stabilizing composition.

7. The stabilizing composition according to claim 1, wherein the cinnamic acid derivative is selected from the group consisting of pentaerythrityl tetra-di-t-butyl hydroxyhydrocinnamate, octadecyl di-t-butyl-4-hydroxyhydrocinnamate, and mixtures thereof.

8. The stabilizing composition according to claim 1, wherein the antioxidant is present in an amount ranging from 0.05 wt. % to 2 wt. %, relative to the total weight of the stabilizing composition.

9. The stabilizing composition according to claim 1, further comprising at least one chelating agent.

10. The stabilizing composition according to claim 9, wherein the chelating agent is selected from the group consisting of sodium citrate, disodium EDTA, tetrasodium EDTA, tetrasodium GLDA, trisodium EDDS, sodium phytate, potassium phytate, and mixtures thereof.

11. The stabilizing composition according to claim 9, wherein the chelating agent is present in an amount ranging from 0.001 wt. % to 1 wt. %, relative to the total weight of the stabilizing composition.

12. The stabilizing composition according to claim 1, further comprising at least one surfactant.

13. The stabilizing composition according to claim 12, wherein the surfactant is present in an amount ranging from 0.05 wt. % to 5 wt. %, relative to the total weight of the stabilizing composition.

14. A combination, comprising:
(a) at least one oil-soluble or oil-dispersible active ingredient; and
(b) the stabilizing composition according to claim 1.

15. The combination according to claim 14, wherein the oil-soluble or oil-dispersible active ingredient is selected from the group consisting of retinol and derivatives thereof.

16. The combination according to claim 14, wherein the oil-soluble or oil-dispersible active ingredient is present in an amount ranging from 0.01 wt. % to 3 wt. %, relative to the total weight of the combination.

17. The stabilizing composition according to claim 1, being in the form of an oil-in-water emulsion having a lamellar structure or a lamellar structured-phase.

18. A non-therapeutic method for caring for and/or making up keratin materials, comprising applying the combination according to claim 14 to the keratin materials.

19. The combination according to claim 14, being in the form of an oil-in-water emulsion having a lamellar structure or a lamellar structured-phase.
